(19) United States
(12) Patent Application Publication
(10) Pub. No.: US 2025/0057926 A1
(43) Pub. Date: Feb. 20, 2025

(54) A METHOD FOR REDUCING AMMONIA EMISSION OF ANIMALS

(71) Applicants: NOVOZYMES A/S, Bagsvaerd (DK); DSM IP ASSETS B.V., Heerlen (NL)

(72) Inventors: Aurélia Anne Catherine Charlotte SÉON ÉP. SIMON, Kaiseraugst (CH); Jose-Otavio SORBARA, Kaiseraugst (CH); Qian ZHANG, Kaiseraugst (CH)

(21) Appl. No.: 18/722,445

(22) PCT Filed: Dec. 8, 2022
(86) PCT No.: PCT/EP2022/084974

§ 371 Date: Jun. 20, 2024

(30) Foreign Application Priority Data
Dec. 23, 2021 (WO) PCT/CN2021/140704

(51) Int. Cl. A61K 38/48; A23K 20/189; A61P 1/14

(52) U.S. Cl. CPC A61K 38/482; A23K 20/189; A61P 1/14

(57) ABSTRACT

The present invention provides a method for reducing ammonia emission of animals comprising administering to the animals one or more proteolytic enzymes, i.e., proteases. The present invention also provides a feed composition suitable for the above methods and use thereof.

### TECHNICAL FIELD

The present invention is related to a method for reducing ammonia emission of animals.

### BACKGROUND OF THE INVENTION

Ammonia emissions in animal breeding industry represents one of several pain points as health or welfare issues affecting animal growth performance and farmers health. Techniques to reduce ammonia emission of animals while meeting nutritional needs of the animals take into consideration combination of nutritional management, reduction of dietary crude protein, addition of essential amino acids and/or addition of feed enzymes.

Protease enzymes are wildly used in animal feeds because they hydrolyze proteins in the feeds and break them down into more usable peptides. Alternative protein sources to soybean meal (SBM), such as Cottonseed Meal (CSM) and Corn Distillers Dried Grains with Solubles (DDGS) can have lower quality protein levels and reduced digestibility in animals. Supplementing protease helps the animals that lack adequate levels of endogenous enzymes to digest proteins in the diet, which reduces the flow of undigested protein and other anti-nutritionals entering the large intestine.

WO 95/02044 A discloses proteases derived from Aspergillus aculeatus, as well as the use in animal feed thereof. WO 01/58276 A discloses acid-stable proteases from the genus Nocardiopsis and their use in animal feed. WO 2019/043191 A1 discloses an animal feed or animal feed additive comprising proteases which can improve animal performance and the nutritional value of the animal feed.

Surprisingly, the inventors of the present invention discovered that proteases provide benefit in reducing ammonia emission of animals.

### SUMMARY OF THE INVENTION

Accordingly, the present invention provides a method for reducing ammonia emission of an animal comprising administering to the animal one or more proteolytic enzymes, i.e., proteases.

The present invention also provides a feed composition, a feed additive and an animal feed comprising one or more proteolytic enzymes, i.e., proteases, for reducing ammonia emission of an animal, and use thereof.

### DETAILED DESCRIPTION OF THE INVENTION

In the present invention, the term “animal” or “animals” refers to any animal except humans. Examples of animals include but are not limited to pigs or swine (including but not limited to piglets, growing pigs and sows); poultry such as turkeys, ducks, quail, guinea fowl, geese, pigeons (including squabs) and chicken (including but not limited to broiler chickens (referred to herein as broilers), chicks, layer hens (referred to herein as layers)); pets such as cats and dogs; and horses.

In the present invention, the term “animal feed” refers to any compound, preparation, or mixture suitable for or intended for intake by an animal and capable of maintaining life and/or promoting production of the animal without any additional substance being consumed except water.

In the present invention, the term “feed additive” refers to an ingredient or combination of ingredients added to the animal feed, usually used in micro quantities and requires careful handling and mixing. Such ingredient includes but is not limited to vitamins, amino acids, minerals, enzymes, eubiotics, colouring agents, growth improving additives and aroma compounds/flavourings, polyunsaturated fatty acids (PUFAs); reactive oxygen generating species, antioxidants, anti-microbial peptides, anti-fungal polypeptides and mycotoxin management compounds etc..

In the present invention, the term “litter” refers to a mixture of bedding material, excreta, feathers, wasted feed and wasted water of animals such as poultry.

The present invention provides a method for reducing ammonia emission of an animal comprising administering to the animal one or more proteolytic enzyme(s), i.e., protease(s).

The present invention also provides use of one or more proteolytic enzyme(s), i.e., protease(s), for reducing ammonia emission of an animal.

In the present invention, the ammonia emission may be characterized by the amount of ammonia gas emitted from urine, faeces, manure (mixture of urine and feces), excreta and/or litter of animals.

In the present invention, the reduction is compared to the animals to which any protease is not administered (herein referred to as the control). Preferably, the ammonia emission of animals is reduced by at least 5%, such as by at least 8%, at least 10%, at least 12%, at least 15%, at least 18% or at least 20% compared to the control.

In the present invention, the proteolytic enzyme or protease catabolizes peptide bonds in proteins breaking them down into fragments of amino acid chains, or peptides.

Proteases are classified according to their catalytic mechanism into the following groups: serine proteases(S), cysteine proteases (C), aspartic proteases (A), metalloproteases (M), and unknown, or as yet unclassified, proteases (U) (see Handbook of Proteolytic Enzymes, A. J. Barrett, N. D. Rawlings, J. F. Woessner (eds), Academic Press (1998)). The protease according to the present invention is a serine protease, preferably an acid stable serine protease, and more preferably a S8 protease, such as those disclosed in WO 2019/043191 A1.

There are no limitations on the origin of the protease according to the invention. Thus, the term protease includes not only natural or wild-type proteases, but also any mutants, variants, fragments etc. thereof exhibiting protease activity, as well as synthetic proteases, such as shuffled proteases, and consensus proteases. Such genetically engineered proteases can be prepared as is generally known in the art, e.g. by site-directed mutagenesis, by PCR (using a PCR fragment containing the desired mutation as one of the primers in the PCR reactions), or by random mutagenesis. The preparation of consensus proteins is described in e.g. EP 0 897 985.

Preferably, the protease according to the present invention is a microbial protease, the term microbial indicating that the protease is derived from, or originates from a microorganism, or is an analogue, a fragment, a variant, a mutant, or a synthetic protease derived from a microorganism. It may be produced or expressed in the original wild-type microbial strain, in another microbial strain, or in a plant; i.e. the term covers the expression of wild-type, naturally occurring proteases, as well as expression in any host of recombinant, genetically engineered or synthetic proteases.

Examples of the microorganism are bacteria, e.g. bacteria of the Family: Nocardiopsaceae, e.g. of the Genus: Nocardiopsis, e.g. Nocardiopsis sp. NRRL 18262, and Nocardiopsis alba; bacteria of the family Bacillaceae, e.g. of the genus Bacillus, e.g. Bacillus horneckiae and Bacillus sp.; and bacteria of the families Pianococcaeae and Paenibacillaceae; and mutants or variants thereof.

Preferred protease according to the present invention is an acid stable serine protease obtained or obtainable from the Genus: Nocardiopsis, such as those derived from Nocardiopsis dassonvillei DSM 43235 (A1918L1), Nocardiopsis prasina DSM 15649 (NN018335L1), Nocardiopsis prasina (previously alba) DSM 14010 (NN18140L1), Nocardiopsis sp. DSM 16424 (NN018704L2), Nocardiopsis alkaliphila DSM 44657 (NN019340L2) and Nocardiopsis lucentensis DSM 44048 (NN019002L2); or the Genus: Bacillus, e.g. Bacillus horneckiae, Bacillus sp TY145, Bacillus sp-13380, Bacillus idriensis, Bacillus sp-62451 and Bacillus oceanisediminis; as well as homologous proteases. Commercially available serine proteases are Ronozyme®ProAct (DSM Nutritional Products AG, Switzerland), ProAct 360™ (DSM Nutritional Products Ltd., Switzerland), and AxtraPro (Dupont, USA)

In the present invention, the protease, besides being acid-stable, may also be thermostable. The term thermostable means for proteases the temperature optimum is at least 50° C., 52° C., 54° C., 56° C., 58° C., 60° C., 62° C., 64° C., 66° C., 68° C., or at least 70° C.

According to the present invention, the protease may be provided in a dosage of between 1,000 units/kg animal feed and 1,000,000 units/kg animal feed, for example in one of the following amounts (dosage ranges): 1,000, 2,000, 4,000, 6,000, 8,000, 10,000, 15,000, 20,000, 30,000, 50,000, 80,000, 100,000, 150,000, 200,000, 250,000, 300,000, 500,000, 600,000, 800,000, 1,000,000 units/kg animal feed. One protease unit (PROT) is the amount of enzyme that releases 1 μmol of p-nitroaniline (pNA) from 1 mM substrate (such as N-succinyl-Ala-Ala-Pro-Phe-pNA) per minute at pH 9.0 and 37° C.

Protease activity can be measured using any assay, in which a substrate is employed, that includes peptide bonds relevant for the specificity of the protease in question. Examples of protease substrates are casein, and pNA-substrates, such as Suc-AAPF-pNA (available e.g. from Sigma S-7388) and N-succinyl-Ala-Ala-Pro-Phe-pNA (Bachem AG, Switzerland). WO2021/180539A1 describes suitable protease assays (see examples 1-4).

In the present invention, the one or more proteolytic enzyme(s), i.e., protease(s) may be formulated in the form of a feed composition or a feed additive (premix) for administering to animals.

Accordingly, the present invention also provides a feed composition or a feed additive comprising one or more proteolytic enzyme(s), i.e., protease(s), as defined above for reducing ammonia emission of an animal.

In the present invention, the feed composition, the feed additive and/or the components such as the protease contains therein may be formulated as a liquid formulation or a solid formulation, and thus May contains one or more formulating agents.

The formulating agents may be selected from the group consisting of polyol such as glycerol, sorbitol, ethylene glycol, diethylene glycol, triethylene glycol, 1,2-propylene glycol, 1,3-propylene glycol, dipropylene glycol and polyethylene glycol (PEG); a salt such as organic or inorganic zinc, sodium, potassium, calcium or magnesium salts (for example, magnesium sulfate, calcium acetate, calcium benzoate, calcium carbonate, calcium chloride, calcium citrate, calcium sorbate, calcium sulfate, potassium acetate, potassium benzoate, potassium carbonate, potassium chloride, potassium citrate, potassium sorbate, potassium sulfate, sodium acetate, sodium benzoate, sodium carbonate, sodium chloride, sodium citrate, sodium sulfate, zinc acetate, zinc benzoate, zinc carbonate, zinc chloride, zinc citrate, zinc sorbate and zinc sulfate); and starch or a sugar or sugar derivative such as sucrose, dextrin, glucose, lactose and sorbitol; small organic molecules, flour, cellulose and minerals and clay minerals (also known as hydrous aluminum phyllosilicates such as kaolinite or kaolin).

The feed composition or the feed additive according to the present invention may also comprise one or more emulsifying agents. The emulsifying agents may be selected advantageously from the group consisting of polyglycerol esters of fatty acids such as esterified ricinoleic acid or propylene glycol esters of fatty acids, saccharo-esters or saccharo-glycerides, polyethylene glycol, lecithins, etc..

Optionally, the feed composition or the feed additive of the present invention may further contain antimicrobial peptides; polyunsaturated fatty acids (PUFAs); reactive oxygen generating species; at least one enzyme, and fat- and water-soluble vitamins, as well as minerals.

Examples of antimicrobial peptides (AMP's) are CAP18, leucocin A, protegrin-1, thanatin, defensin, lactoferrin, lactoferricin, and ovispirin such as novispirin (Robert Lehrer, 2000), plectasins, and statins.

Examples of polyunsaturated fatty acids are C₁₈-, C₂₀- and C₂₂-polyunsaturated fatty acids, such as arachidonic acid, docosohexaenoic acid, eicosapentaenoic acid and gamma-linoleic acid.

Examples of reactive oxygen generating species are chemicals such as perborate, persulphate, or percarbonate; and enzymes such as an oxidase, an oxygenase or a syntethase.

Examples of enzyme are phytase (EC 3.1.3.8 or 3.1.3.26), galactanase (EC 3.2.1.89), alpha-galactosidase (EC 3.2.1.22), phospholipase A 1 (EC 3.1.1.32), phospholipase A2 (EC 3.1.1.4), lysophospholipase (EC 3.1.1.5), phospholipase C (EC 3.1.4.3), and/or phospholipase D (EC 3.1.4.4).

Examples of fat-soluble vitamins include but are not limited to vitamin A, vitamin D₃, and vitamin K, e.g. vitamin K₃.

Examples of water-soluble vitamins include but are not limited to vitamin B12, biotin and choline, vitamin B₁, vitamin B₂, vitamin B₆, niacin, folic acid and panthothenate, e.g. Ca-D-panthothenate.

Examples of minerals include but are not limited to calcium, phosphorus, sodium, potassium, magnesium, chlorine, iodine, iron, manganese, copper, molybdenum, cobalt and zinc. Common mineral supplements in feed are: limestone, Bone meal, oyster shell, sodium chloride, dicalcium phosphate, manganese sulphate, potassium iodide, and superphosphate. Sources of minerals include meat scraps, fish meal, milk products, ground limestone (calcium), ground oyster shells (calcium), dicalcium phosphate (calcium, phosphorus), defluorinated rock phosphate (phosphorus, calcium), steamed bone meal (phosphorus, calcium), salt (sodium, chlorine, iodine), manganese sulfate (manganese), manganese oxide (manganese), zinc carbonate (zinc), zinc oxide (zinc).

In the feed composition or the feed additive of the present invention, the one or more proteolytic enzyme(s), i.e., protease(s), may be contained in a total amount of from 0.5 wt % to 50 wt %, preferably from 1 wt % to 30 wt %, more preferably from 2 wt % to 20 wt %, and the most preferably from 5 wt % to 15 wt %, based on the weight of the feed composition or the feed additive of the present invention.

As anticipated by a person skilled in the art, the one or more proteolytic enzyme(s), i.e., protease(s), the feed composition or the feed additive according to the present invention may be finally incorporated into an animal feed.

Accordingly, the present invention further provides an animal feed which comprises the one or more proteolytic enzyme(s), i.e., protease(s), the feed composition or the feed additive, as defined above, for reducing ammonia emission of an animal.

In the animal feed according to the present invention, the protease may be provided in a dosage of between 1,000 units/kg animal feed and 1,000,000 units/kg animal feed, for example in one of the following amounts (dosage ranges): 1,000, 2,000, 4,000, 6,000, 8,000, 10,000, 15,000, 20,000, 30,000, 50,000, 80,000, 100,000, 150,000, 200,000, 250,000, 300,000, 500,000, 600,000, 800,000, 1,000,000 units/kg animal feed.

Preferably, the animal feed according to the present invention is an animal diet based on soy-bean meal, corn and/or wheat.

As also anticipated by any person skilled in the art, the animal feed according to the present invention may further include any number of components typical for an animal feed, such as proteins, fats and additional additives.

Examples of suitable types of proteins that can be included in the feed include, but are not limited to, meat scraps (lysine), fish meal (lysine, methionine), poultry by-product meal (tryptophan, lysine), blood meal, liver and glandular meal, feather meal (hydrolyzed), animal tankage, milk products, cottonseed meal, peanut meal, soybean meal, sesame meal, sunflower seed meal.

Most feed ingredients (maize, barley, safflower, milo, wheat, rice, bran, etc.) contain approximately 2-5% fat and linoleic acid. Sources of fats include animal tallow (beef), lard, corn oil, and other vegetable oils.

Additional additives include but are not limited to antioxidants like BHT (Butylated hydroxytoluene), santoquin, ethoxyquin, butylated hydroxyanisode and diphenyl paraphenyl diamine; pellet binders such as sodium bentonite (clay), liquid or solid by-products of the wood pulp industry, molasses, and guarmeal; coloring agents such as xanthophylls, synthetic carotinoid, and canthaxanthin; probiotics such as strains of lactobacillus and streptococcus; and/or antibiotics such as penicillin, streptomycin, tetracyclines, and aureomycin.

Any person skilled in the art are familiar with the particular recipes for making the feed for particular types of animals and can be prepared in similar formulations when adding an effective amount of the composition or the animal feed additive according to the present invention.

The present invention will be further illustrated by the following examples.

### EXAMPLES

### Example 1: In-Vivo Trial

### Materials and Methods

Day-old male broiler chickens (Cobb 500) were housed in floor pen covered with wood shavings in an environmentally controlled room. The room temperature and lighting program was adapted according to the age specific requirements of the chickens. Feed and water were provided ad libitum.

At the arrival, birds were sorted by body weight, transferred and randomly allocated to one of 2 dietary treatments, which were supplemented with protease product ProAct 360™ (Protease, DSM Nutritional Product, Switzerland) at 0 and 30,000 NFP/kg (Table 1). For each treatment, there were 12 cages with 40 birds per cage.

TABLE 1
Dietary treatments

| Treat | Description | Protease dosing |
|---|---|---|
| A | Control | 0 |
| B | Protease | 30,000 NFP/kg |

The composition and ingredients of the experimental diets had been shown in Table 2.

TABLE 2
Composition and ingredients of the experimental diets

| | Starter | Grower | Finisher |
|---|---|---|---|
| Ingredients (%) | (d 0-14) | (d 14-28) | (d 28-42) |
| Corn | 57.61 | 60.67 | 65.94 |
| Soybean meal | 36.60 | 33.70 | 28.85 |
| Soybean oil | 1.50 | 1.80 | 1.82 |
| NaCl | 0.26 | 0.25 | 0.23 |
| NaHCO₃ | 0.30 | 0.30 | 0.29 |
| DL-Met | 0.28 | 0.26 | 0.24 |
| L-Lys · HCl | 0.14 | 0.12 | 0.12 |
| Limestone | 0.44 | 0.36 | 0.32 |
| DCP dihydrate | 2.33 | 2.00 | 1.65 |
| ¹Vit-Min Premix | 0.50 | 0.50 | 0.50 |
| Lasalocid 20% | 0.045 | 0.045 | 0.045 |
| Total | 100.00 | 100.00 | 100.00 |

¹Broiler premix provided (per kg of diet): Vitamin A 10000 IU, Vitamin D₃ 2240 IU, 25-OH-D3 69 μg, Vitamin E 50 IU, Vitamin K₃ 3 mg, Vitamin B₁ 2 mg, Vitamin B₂ 7 mg, Vitamin B₆ 4 mg, Vitamin B₁₂ 0.02 mg, Biotin 0.25 mg, Folic acid 2 mg, Niacinamide 60 mg, D-pantothenic acid 12 mg, Fe 40 mg, Cu 15 mg, Mn 110 mg, Zn 90 mg, 1 0.5 mg, Se 0.25 mg, Choline 400 mg.

### Measurements

Ammonia flux measurements from the litter were measured within 4 pens per treatment at the end of the trial: 48 measures of NH₃ were done at litter surface within each pen at 3 locations at 4 different times: time zero and at 60, 120 and 180 seconds using Dräger X8000 device.

### Statistical Analysis

Data were subjected to a one-way analysis of variance, using the StatGraphics Centurion XVI statistical software package (Manugistics, Rockwille, MD) followed by the Tukey HSD multiple comparison procedure to assess differences among means in treatment groups. One cage was defined as the experimental and statistical unit. The effects of treatment were considered significant at p<0.05.

### Results

In the measurement of ammonia emission at litter surface, decrease of ammonia (NH₃) concentration (by 32%) was observed at day 41, at litter surface of birds fed diet containing Protease from 43 ppm to 29 ppm as shown in table 3.

TABLE 3
Effect of protease on ammonia emission from poultry litter at day 41

| | Ammonia emission ppm (Mean ± SEM) |
|---|---|
| A: Control A | 42.5 ± 4 |
| B: A + Protease | 28.7 ± 4 |
| P-Value | 0.019 |

### CONCLUSION

Ammonia emission from poultry litter were significantly reduced (by 32%) when animals were supplemented with Protease compare to control without protease.

1. A method for reducing ammonia emission of an animal comprising administering to the animal one or more proteolytic enzyme(s), i.e., protease(s).

2. The method of claim 1, wherein the protease is a serine protease, preferably an acid stable serine protease, and more preferably a S8 protease.

3. The method of claim 1, wherein the protease is a microbial protease.

4. The method of claim 1, wherein the protease is derived from a microorganism such as bacteria, e.g. bacteria of the Family: Nocardiopsaceae, e.g. of the Genus: Nocardiopsis, e.g. Nocardiopsis sp. NRRL 18262, and Nocardiopsis alba; bacteria of the family Bacillaceae, e.g. of the genus Bacillus, e.g. Bacillus horneckiae and Bacillus sp.; and bacteria of the families Pianococcaeae and Paenibacillaceae; and mutants or variants thereof.

5. The method of claim 1, wherein the protease is an acid stable serine protease obtained or obtainable from the Genus: Nocardiopsis, such as those derived from Nocardiopsis dassonvillei DSM 43235 (A1918L1), Nocardiopsis prasina DSM 15649 (NN018335L1), Nocardiopsis prasina (previously alba) DSM 14010 (NN18140L1), Nocardiopsis sp. DSM 16424 (NN018704L2), Nocardiopsis alkaliphila DSM 44657 (NN019340L2) and Nocardiopsis lucentensis DSM 44048 (NN019002L2); or the Genus: Bacillus, e.g. Bacillus horneckiae, Bacillus sp TY145, Bacillus sp-13380, Bacillus idriensis, Bacillus sp-62451 and Bacillus oceanisediminis; as well as homologous proteases.

6. The method of claim 1, wherein the protease is acid-stable and thermostable.

7. The method of claim 1, wherein the protease is provided in a dosage of between 1,000 units/kg animal feed and 1,000,000 units/kg animal feed, for example in one of the following amounts (dosage ranges): 1,000, 2,000, 4,000, 6,000, 8,000, 10,000, 15,000, 20,000, 30,000, 50,000, 80,000, 100,000, 150,000, 200,000, 250,000, 300,000, 500,000, 600,000, 800,000, 1,000,000 units/kg animal feed.

8. A feed composition, a feed additive or an animal feed comprising one or more proteolytic enzymes, i.e., proteases, for reducing ammonia emission of an animal.

9. The feed composition, a feed additive or an animal feed of claim 8, wherein the protease is contained in a total amount of from 0.5 wt % to 50 wt %, preferably from 1 wt % to 30 wt %, more preferably from 2 wt % to 20 wt %, and the most preferably from 5 wt % to 15 wt %, based on the weight of the feed composition, the feed additive or the animal feed.

10. Use of one or more proteolytic enzymes, i.e., proteases, and a carbohydrase in an animal feed for reducing ammonia emission of an animal.
